(19) United States
(12) Patent Application Publication
(10) Pub. No.: US 2025/0107814 A1
(43) Pub. Date: Apr. 3, 2025

(54) METHOD FOR POSITIONING MEDICAL DEVICE AND MEDICAL DEVICE

(71) Applicant: TERUMO KABUSHIKI KAISHA, Tokyo (JP)

(72) Inventors: Taiga NAKANO, Irvine, CA (US); Kosuke NISHIO, Irvine, CA (US); Cang LAM, Irvine, CA (US); Vivian TRAN, Irvine, CA (US)

(73) Assignee: TERUMO KABUSHIKI KAISHA, Tokyo (JP)

(21) Appl. No.: 18/898,837

(22) Filed: Sep. 27, 2024

(30) Foreign Application Priority Data
Sep. 29, 2023 (JP) 2023-169509

(51) Int. Cl. A61B 17/22; A61B 17/00; A61B 17/32

(52) U.S. Cl. CPC A61B 17/22; A61B 17/32; A61B 2017/00141; A61B 2017/00398; A61B 2017/00778

(57) ABSTRACT

Provided is a method for positioning a medical device for cutting an object in a blood vessel, the medical device including: a drive shaft that is rotatable; a cutter disposed on the distal side with respect to the drive shaft; a drive unit that rotates the drive shaft; and an outer tube shaft that rotatably accommodates the drive shaft. The method includes bringing the cutter close to a lesion within the blood vessel, and then, rotating the cutter at a preset first rotation speed, determining whether or not a stop condition of the prior operation at the first rotation speed is satisfied, and rotating the cutter at a preset second rotation speed of 10,000 rpm or more and 150,000 rpm or less after it is determined that the stop condition of the prior operation is

[symbol]

### CROSS-REFERENCES TO RELATED APPLICATION

This application claims priority to Japanese Patent Application No. 2023-169509 filed on Sep. 29, 2023, the entire content of which is incorporated herein by reference.

### TECHNOLOGICAL FIELD

The present invention generally relates to a method for positioning a medical device for cutting an object in a body lumen and a medical device.

### BACKGROUND DISCUSSION

Treatment methods for treating a stenosed part due to plaque, thrombus, or the like in a blood vessel include, for example, a method for expanding the blood vessel with a balloon and a method for placing a meshed or coiled stent in the blood vessel as a support of the blood vessel. However, with these methods, it is difficult to treat a stenosed part hardened due to calcification or a stenosed part located in a bifurcation of a blood vessel. As a device that can provide treatment even in such a case, an atherectomy device that cuts and removes a lesion has been proposed. An example of such a device is disclosed in U.S. Pat. No. 8,628,549.

An atherectomy device includes a cutter that rotates at a high speed at a distal end part of a drive shaft which is rotatable inside an outer tube shaft, the atherectomy device shearing or crushing thrombus, plaque, or the like in a blood vessel to remove the same. In particular, the atherectomy device in which the distal end part of the outer tube shaft is shaped so that it is possible to adjust the orientation of the distal end of the outer tube shaft, and can thus selectively treat plaque or the like present non-uniformly on the inner wall of the blood vessel.

### SUMMARY

The structure in the blood vessel varies, and the blood vessel may often meander and have many irregularities. Under such conditions, after the outer tube shaft of the atherectomy device is positioned in terms of orientation, a twist may accumulate in the outer tube shaft, and a phenomenon may occur in which, triggered by the vibration caused by high-speed rotation of the drive shaft or a force for moving the outer tube shaft back and forth during treatment, the orientation of the outer tube shaft greatly deviates. When this phenomenon occurs, the site to be originally treated cannot be treated, and further, the vessel wall not to be treated may be damaged.

The method disclosed here makes it possible to position a cutter in an appropriate orientation, and a medical device.

(1) A method involves positioning a medical device in a blood vessel and cutting an object in the blood vessel. The medical device includes a drive shaft that is rotatable, a cutter disposed on a distal side with respect to the drive shaft, a drive unit that rotates the drive shaft, and an outer tube shaft that rotatably accommodates the drive shaft. The method includes: bringing the cutter close to a lesion within the blood vessel, and then, rotating the cutter at a first rotation speed that is set in advance; determining whether or not a stop condition of a prior operation at the first rotation speed is satisfied; and rotating the cutter at a second rotation speed that is set in advance after it is determined that the stop condition of the prior operation is satisfied, the second rotation speed being 10,000 rpm or more and 150,000 rpm or less.

With the method for positioning the medical device according to (1), the twist of the drive shaft and the outer tube shaft can be eliminated by the prior operation, whereby the cutter can be positioned in an appropriate orientation before performing a cutting operation.

(2) The method for positioning a medical device according to (1) may further include a step for reducing or stopping the rotation of the cutter after it is determined that the stop condition of the prior operation is satisfied and before the cutter is rotated at the second rotation speed. With this configuration, it is possible to confirm whether or not the twist of the drive shaft and the outer tube shaft has been eliminated by the prior operation before a main operation for cutting is performed.

(3) The method for positioning a medical device according to (1) or (2), wherein the first rotation speed may be 400 rpm or more and 10,000 rpm or less. With this configuration, the prior operation is performed at a low speed, which makes the cutter less likely to perform the cutting operation during the prior operation. Thus, safety can be improved.

(4) The method for positioning a medical device according to (1) or (2), wherein the first rotation speed may be 10,000 rpm or more and 60,000 rpm or less. With this configuration, the first rotation speed is substantially the same as the second rotation speed, whereby the adjustment of the rotation speed is facilitated.

(5) The method for positioning a medical device according to any one of (1) to (4), wherein the stop condition of the prior operation may be an elapsed time of 0.01 seconds or more. With this configuration, the stop condition of the prior operation can be easily set so that the twist of the drive shaft and the outer tube shaft can be eliminated.

(6) The method for positioning a medical device according to any one of (1) to (4), wherein the stop condition of the prior operation may be a number of rotations of the cutter that is five or more. With this configuration, the stop condition of the prior operation can be easily set so that the twist of the drive shaft and the outer tube shaft can be eliminated.

(7) The method for positioning a medical device according to any one of (1) to (6), wherein, in the step for rotating the cutter at the first rotation speed, the cutter may be rotated at the first rotation speed after the cutter is oriented to the lesion within the blood vessel. The twist of the drive shaft and the outer tube shaft is eliminated after the cutter is oriented to the lesion in the blood vessel, whereby it is easy to recognize the elimination of the twist.

(8) The method for positioning a medical device according to any one of (1) to (6), wherein, in the step for rotating the cutter at the first rotation speed, the cutter may be rotated at the first rotation speed as the cutter is oriented to the lesion within the blood vessel. The twist of the drive shaft and the outer tube shaft is eliminated as the cutter is oriented to the lesion in the blood vessel, whereby it is easy to appropriately adjust the position and the orientation of the cutter that have changed after the elimination of the twist.

(9) According to another aspect, a medical device for cutting an object in a blood vessel includes: a drive shaft that is rotatable; a cutter disposed on a distal side with respect to the drive shaft; a drive unit that rotates the drive shaft; an outer tube shaft that rotatably accommodates the drive shaft; and a control unit that controls driving of the drive unit. The control unit is configured to control the drive unit to rotate the cutter at a first rotation speed that is set in advance, determines whether or not a stop condition of a prior operation at the first rotation speed is satisfied, and rotates the cutter at a second rotation speed that is set in advance after determining that the stop condition of the prior operation is satisfied, the second rotation speed being 10,000 rpm or more and 150,000 rpm or less.

The medical device according to (9) can eliminate the twist of the drive shaft and the outer tube shaft by the prior operation, thereby being capable of positioning the cutter in an appropriate orientation before performing a cutting operation.

(10) The medical device according to (9), wherein the first rotation speed may be 400 rpm or more and 10,000 rpm or less. With this configuration, the prior operation is performed at a low speed, which makes the cutter less likely to perform the cutting operation during the prior operation. Thus, safety can be improved.

(11) The medical device according to (9), wherein the first rotation speed may be 10,000 rpm or more and 60,000 rpm or less. With this configuration, the first rotation speed is substantially the same as the second rotation speed, whereby the adjustment of the rotation speed is facilitated.

(12) The medical device according to any one of (9) to (11), wherein the stop condition of the prior operation may be an elapsed time of 0.01 seconds or more and 10 seconds or less. With this configuration, the stop condition of the prior operation can be easily set so that the twist of the drive shaft and the outer tube shaft can be eliminated.

(13) The medical device according to any one of (9) to (11), wherein the stop condition of the prior operation may be a number of rotations of the cutter that is five or more. With this configuration, the stop condition of the prior operation can be easily set so that the twist of the drive shaft and the outer tube shaft can be eliminated.

(14) The medical device according to any one of (9) to (11), wherein a stop condition of main rotation at the second rotation speed may be an elapsed time longer than the time of the prior operation. With this configuration, it is possible to perform the main operation for cutting for sufficient time, whereby appropriate cutting can be performed.

According to another aspect, a method comprises: positioning a cutter in a blood vessel, with the cutter being connected to a rotatable drive shaft so that rotation of the drive shaft results in rotation of the cutter; moving the cutter towards a lesion in the blood vessel; starting rotation of the cutter and continuing rotation of the cutter during a first operation at a first rotation speed through rotation of the drive shaft while the lesion is located distal of the cutter so that the cutter does not cut the lesion; determining that a stop condition of the first operation at the first rotation speed is satisfied; and rotating the cutter at a preset second rotation speed through rotation of the drive shaft while the cutter is in contact with the lesion so that the lesion is cut by rotation of the cutter at the second rotation speed, the rotating of the cutter at the preset second rotation speed occurring after determining that the stop condition of the first operation at the first rotation speed has been satisfied.

### BRIEF DESCRIPTION OF THE DRAWINGS

FIG. 1 is a plan view illustrating a medical device.

FIG. 2 is a cross-sectional view illustrating a distal end part of the medical device.

FIG. 3 is a plan view illustrating an inner layer and an outer layer of the medical device in the vicinity of the distal end part, FIG. 3 including a plan view of a drive shaft and a conveyance coil and a cross-sectional view of other components.

FIG. 4 is a configuration diagram of the medical device.

FIG. 5 is a schematic diagram illustrating a state in which the medical device removes a lesion.

FIG. 6 is a flowchart illustrating a flow of control in a control unit.

FIG. 7 is a plan view illustrating a modification of an operation instruction portion provided on a handle.

### DETAILED DESCRIPTION

An embodiment of the medical device disclosed here, representing one example of the new medical device, will be described below with reference to the drawings. The size and ratio of each member in the drawings may be exaggerated for convenience of description and may be different from the actual size and ratio. In addition, in the present specification, a side of a medical device 10 to be inserted into a living body is referred to as a “distal side” and a side to be operated is referred to as a “proximal side”.

The medical device 10 according to the present embodiment is inserted into a blood vessel in acute lower limb ischemia or deep-vein thrombosis, and used for a treatment including destroying (breaking-up or breaking-apart) and removing an object such as a thrombus, plaque, atheroma, and a calcified lesion. The object to be removed is not necessarily limited to a thrombus, plaque, atheroma, and a calcified lesion, and may be any object that can be present in the body lumen.

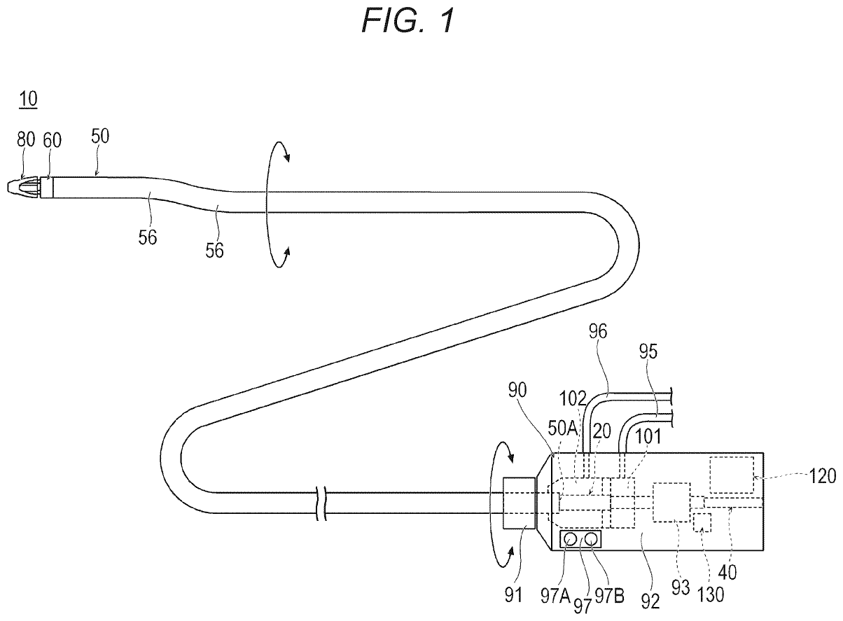

As illustrated in FIGS. 1 to 4, the medical device 10 includes an elongated drive shaft 20, a conveyance coil 30 that conveys an object, an outer tubular shaft 50 that accommodates the drive shaft 20 and the conveyance coil 30, a cutter 80 that cuts an object such as thrombus, a rotation shaft 70 that connects the cutter 80 and the drive shaft 20, a distal bearing 60 that rotatably supports the rotation shaft 70, a guide wire lumen tube 40 disposed inside the drive shaft 20, a handle 90, a control unit 120 that controls the operation of the medical device 10, and a rotation detection unit 130.

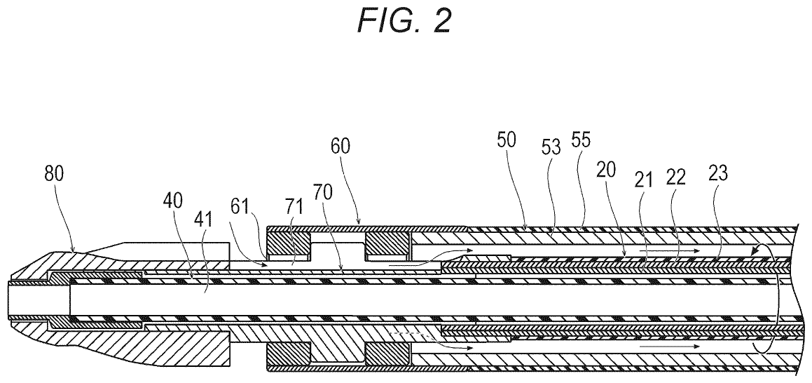

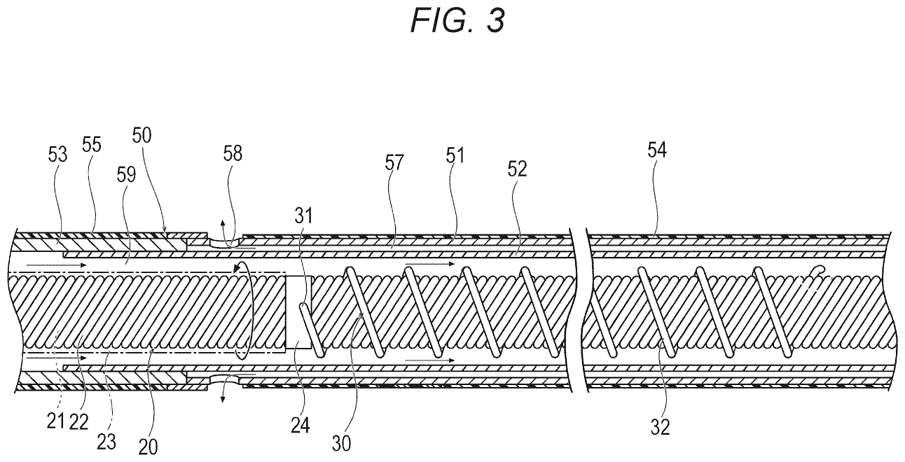

As illustrated in FIGS. 2 and 3, the outer tubular shaft 50 includes an outer layer 51, an inner layer 52, a shaft distal portion 53, a first covering tube 54 in close contact with the outer peripheral surface of the outer layer 51, and a second covering tube 55 in close contact with the outer peripheral surface of the shaft distal portion 53.

The drive shaft 20 is a multilayer coil in which coils are layered. The drive shaft 20 is flexible and has a property of transmitting a drive torque applied from the proximal side to the distal side. The drive shaft 20 is rotatable inside the outer tubular shaft 50. The drive shaft 20 includes an inner coil 21, an outer coil 22 surrounding the outside of the inner coil 21, and a distal protecting tube 23 that covers the outer peripheral surface of the outer coil 22 at the distal end section. The drive shaft 20 may be a multilayer coil including three or more layers.

The outer coil 22 is wound in a direction in which the helix of a wire material constituting the outer coil 22 is tightened to reduce the diameter when the drive shaft 20 rotates in a predetermined rotation direction (rotation direction of the drive shaft 20 when the medical device 10 is used for cutting and conveyance) in response to a drive torque from the proximal side. That is, the wire material of the outer coil 22 is wound in the direction opposite to the predetermined rotation direction toward the distal side when viewed from the proximal side. The outer coil 22 has characteristics of extending along the axis of the outer coil 22 while decreasing in diameter in response to the drive torque from the proximal side during rotation of the drive shaft 20 in the predetermined rotation direction. The outer coil 22 may be a single-wire coil formed by winding a single wire material or a multi-wire coil formed by winding multiple wire materials.

The inner coil 21 is wound in a direction in which the helix of a wire material constituting the inner coil 21 is loosened to increase the diameter when the drive shaft 20 rotates in the predetermined rotation direction in response to a drive torque from the proximal side. That is, the wire material of the inner coil 21 is wound in the predetermined rotation direction toward the distal side when viewed from the proximal side. The inner coil 21 has characteristics of contracting along the axis of the inner coil 21 while increasing in diameter in response to the drive torque from the proximal side during rotation of the drive shaft 20 in the predetermined rotation direction. The inner coil 21 may be a single-wire coil formed by winding a single wire material or a multi-wire coil formed by winding multiple wire materials.

The outer coil 22 is disposed in close contact with the outer peripheral surface of the inner coil 21. Therefore, when the drive shaft 20 rotates in the predetermined rotation direction, the inner coil 21 is going to contract while increasing in diameter, and the outer coil 22 is going to extend while reducing in diameter, so that the displacement of the outer coil 22 and the inner coil 21 in the radial direction and the axial direction is canceled out. Thus, the multilayer coil including the outer coil 22 and the inner coil 21 can reduce deformation in the radial direction and the axial direction when the drive shaft 20 rotates in the predetermined rotation direction. The action of the outer coil 22 is exerted more strongly than the action of the inner coil 21, because the inner coil 21 has a smaller coil radius than the outer coil 22. Therefore, the drive shaft 20 slightly contracts in the radial direction and slightly extends in the axial direction when the drive shaft 20 rotates in the predetermined rotation direction.

As an example, the outer coil 22 is a multi-wire coil formed by helically and tightly winding sixteen wire materials (metallic material). The inner coil 21 is a multi-wire coil formed by helically and tightly winding eight wire materials. The number of wire materials of the outer coil 22 and the inner coil 21 is not particularly limited. The cross-sectional shape of the wire material of each of the outer coil 22 and the inner coil 21 is, for example, circular, but is not particularly limited thereto, and may be elliptical, rectangular, square, triangular, or the like.

The drive shaft 20 may be a single-layer coil. In this case, when the wire material of the single-layer coil is wound in the direction opposite to the predetermined rotation direction toward the distal side as viewed from the proximal side, the drive shaft 20 extends along the axis while reducing in diameter in response to the drive torque from the proximal side. When the wire material of the single-layer coil is wound in the predetermined rotation direction toward the distal side as viewed from the proximal side, the drive shaft 20 contracts along the axis while increasing in diameter in response to the drive torque from the proximal side.

The drive shaft 20 has a ring-shaped or annular integrated portion 24 on the proximal side with respect to the shaft distal portion 53. The integrated portion 24 is formed by melting and joining the wire materials arranged in the circumferential direction of the outer coil 22 by welding and integrating the joined wire materials in the circumferential direction. In the integrated portion 24, not only the outer coil 22 but also at least an outer part of the inner coil 21 is preferably integrated, but the configuration is not limited thereto. The integrated portion 24 reduces the flexibility of the drive shaft 20, and thus, is preferably not too long in the axial direction.

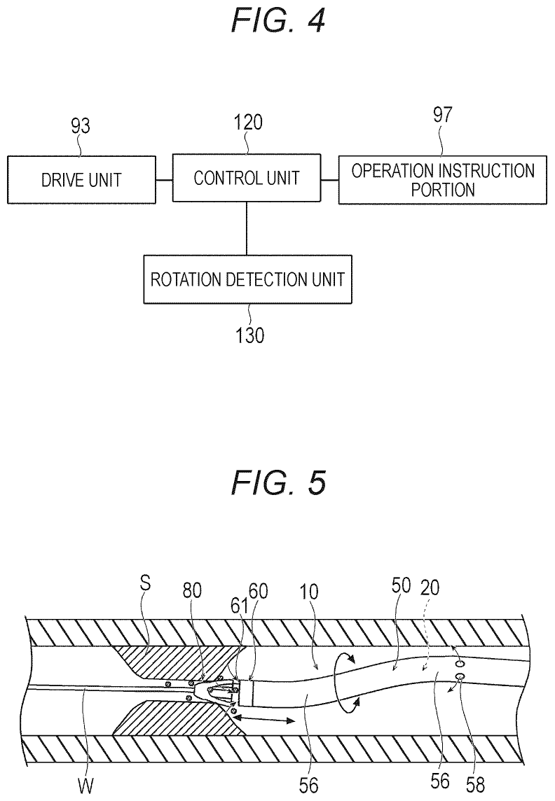

As illustrated in FIGS. 3 and 4, the conveyance coil 30 is disposed so as to wrap around (helically wrap around) the outer periphery of the drive shaft 20. The conveyance coil 30 is partially in contact with the outer peripheral surface of the drive shaft 20, but may be placed with a clearance. The conveyance coil 30 is formed by loosely winding the wire material constituting the conveyance coil 30 at intervals along the axial direction of the conveyance coil 30. That is, as shown in FIG. 3, the conveyance coil 30 is loosely wound in a helical manner with adjacent helical windings axially spaced apart from one another. The conveyance coil 30 is fixed to the drive shaft 20 at a distal end.

The conveyance coil 30 functions as an Archimedean screw (screw pump) when the drive shaft 20 rotates in the predetermined rotation direction, and conveys a liquid or an object in the proximal direction. To this end, the conveyance coil 30 is wound in the predetermined rotation direction toward the distal side when viewed from the proximal side.

Examples of materials that can be preferably used for the drive shaft 20 and the conveyance coil 30 include stainless steel, nitinol (NiTi), Ta, Ti, Pt, Au, W, polyolefin such as polyethylene and polypropylene, polyamide, polyester such as polyethylene terephthalate, fluorine-based polymer such as ethylene-tetrafluoroethylene copolymer (ETFE), polyether ether ketone (PEEK), and polyimide.

As illustrated in FIGS. 2 and 3, the distal protecting tube 23 is a tubular body that covers the outer peripheral surface of the drive shaft 20 on the distal side with respect to the conveyance coil 30. The distal protecting tube 23 is disposed inside the shaft distal portion 53 provided on the outer tubular shaft 50. The distal protecting tube 23 is formed of, for example, a heat shrinkable tube that is reduced in diameter by heating to be in close contact with the drive shaft 20. The distal protecting tube 23 prevents the drive shaft 20 and the shaft distal portion 53 from coming into contact with each other and being damaged by the rotation of the drive shaft 20.

The material of the heat-shrinkable tube is not particularly limited, and is, for example, polyolefin, polyamide, Pebax, polyurethane, or polyethylene terephthalate.

The guide wire lumen tube 40 is a tubular body disposed inside the drive shaft 20 as illustrated in FIG. 2. The guide wire lumen tube 40 is formed with a guide wire lumen 41 through which a guide wire passes. The guide wire lumen tube 40 prevents the guide wire passing through the guide wire lumen 41 from rubbing against the drive shaft 20. The distal end part of the guide wire lumen tube 40 protrudes further to the distal side with respect to the drive shaft 20 and is disposed inside the rotation shaft 70 and the cutter 80. The proximal end part of the guide wire lumen tube 40 is connected to the handle 90.

As illustrated in FIGS. 1 to 4, the outer tubular shaft 50 is a long (elongated) tubular body that accommodates the drive shaft 20. The outer tubular shaft 50 can transmit a torque exerted by an operator on a rotary operation portion 91 fixed to the proximal end part of the outer tubular shaft 50. A first lumen 57 for delivering a liquid such as physiological saline to the distal side is formed between the outer layer 51 and the inner layer 52. At least one side hole 58 penetrating from the inner peripheral surface to the outer peripheral surface is formed at the distal end part of the outer layer 51. The cutter 80 can be oriented to the lesion by rotating the outer tubular shaft 50. The outer tubular shaft 50 has a proximal end opening 50A that opens inside the handle 90 at the proximal end.

It is preferable that the outer layer 51 has flexibility so as to bend in the body lumen and has high torque transmission. Examples of materials that can be used for the outer layer 51 include a material obtained by forming a helical slit or groove by laser processing in a circular tube made of a metal material or a resin material having a certain degree of strength. The material of the outer layer 51 is not particularly limited, and examples thereof that can be preferably used include a metal material such as stainless steel, nitinol (NiTi), Ta, Ti, Pt, Au, and W, and engineering plastics such as ABS resin, polycarbonate (PC), polymethyl methacrylate (PMMA), polyacetal (POM), polyphenyl sulfone (PPSU), polyethylene (PE), carbon fiber, or polyether ether ketone (PEEK).

The first covering tube 54 is a tubular body that is in close contact with the outer peripheral surface of the outer layer 51. The first covering tube 54 suppresses leakage of the liquid in the first lumen 57 from a gap of the helical slit formed in the outer layer 51. The first covering tube 54 is formed of, for example, a heat shrinkable tube that is reduced in diameter by heating to be in close contact with the outer layer 51.

The inner layer 52 is disposed on the inner side of the outer layer 51 with a clearance therebetween. The clearance between the inner layer 52 and the outer layer 51 is the first lumen 57. A second lumen 59 for discharging an object such as a cut thrombus in the proximal direction is formed inside the inner layer 52. The distal end part of the inner layer 52 is fixed to an inner peripheral surface of the shaft distal portion 53 with an adhesive. The proximal end part of the inner layer 52 is located inside the handle 90.

The resin material forming the inner layer 52 desirably has a certain degree of flexibility and low friction, and examples of the material that can be preferably used include polyether ether ketone (PEEK), fluorine-based polymer such as PTFE or ETFE, polymethyl methacrylate (PMMA), polyethylene (PE), polyether block acid copolymer (PEBAX), polyamide, polyimide, and a combination thereof. The inner layer 52 may have a braided reinforcement line. The inner surface of the inner layer 52 is preferably formed of a resin material. This configuration can reduce friction between the inner layer 52 and the conveyance coil 30. As the resin material forming the inner surface of the inner layer 52, polyimide, PTFE, polyamide, or the like can be used. This makes it possible to improve the lubricity while improving the wear resistance of the inner layer 52 and the conveyance coil 30.

As illustrated in FIGS. 2 and 3, the shaft distal portion 53 is located at the distal end part of the outer tubular shaft 50. The shaft distal portion 53 is bent at two bending portions 56 such that the axis of the shaft distal portion 53 at the proximal end part and the axis at the distal end part are shifted from each other. The number of the bending portions 56 may be one or three or more. By rotating the outer tubular shaft 50, the shaft distal portion 53 can orient the cutter 80 toward the lesion and further strongly press the cutter 80 against the lesion. As the material of the shaft distal portion 53, a material like those examples discussed above for the outer layer 51 can be applied, for example.

The second covering tube 55 is a tubular body that is in close contact with the outer peripheral surface of the shaft distal portion 53. The second covering tube 55 is formed of, for example, a heat shrinkable tube that is reduced in diameter by heating to be in close contact with the shaft distal portion 53.

As illustrated in FIG. 2, the distal bearing 60 is fixed to the distal end part of the shaft distal portion 53 and protrudes to the distal side beyond the shaft distal portion 53. The distal bearing 60 supports the rotation shaft 70 fixed to the distal end part of the outer tubular shaft 50 in a rotatable manner. The distal bearing 60 is fixed to the distal end part of the shaft distal portion 53. The distal bearing 60 has, at the distal end, a distal end opening 61 through which an object such as a cut thrombus, blood, and liquid discharged through the side hole 58 is conveyed and delivered into the second lumen 59. The distal end of the distal bearing 60 is positioned proximal to the cutter 80.

The rotation shaft 70 is rotatably supported by the distal bearing 60 fixed to the distal end part of the outer tubular shaft 50. The proximal end part of the rotation shaft 70 is fixed to the distal end part of the drive shaft 20, and the distal end part of the rotation shaft 70 is fixed to the cutter 80. The rotation shaft 70 is formed with at least one groove-shaped passage 71 extending along the axis (extending in the axial direction). The passage 71 allows an object cut by the cutter 80 to pass in the proximal direction through the inside of the distal bearing 60.

The cutter 80 is a member for cutting and reducing the size of an object such as a thrombus, plaque, and a calcified lesion. Therefore, the term “cut” represents the action of applying a force to an object in contact with the cutter 80 and reducing the size of the object. A method for applying a force during cutting and the shape or type of the cut object are not limited. The cutter 80 is strong enough to cut the above-mentioned object. The cutter 80 is fixed to the outer peripheral surface of the rotation shaft 70 at the distal end part. The cutter 80 has a large number of fine abrasive grains on the surface. Alternatively, the cutter 80 may have a sharp blade.

It is preferable that the material of the cutter 80 is strong enough to cut thrombus, and examples of the material that can be preferably used include stainless steel, Ta, Ti, Pt, Au, W, a shape memory alloy, a supersteel alloy, and a ceramic material. The materials of the distal bearing 60 and the rotation shaft 70 preferably have a certain degree of strength, and the above-mentioned materials applicable to the cutter 80 can be used.

The handle 90 includes a rotary operation portion 91, a casing 92, a drive unit 93, an intake port 95, a liquid delivery port 96, and an operation instruction portion 97 as illustrated in FIG. 1.

The rotary operation portion 91 is connected to the distal end part of the casing 92 in a rotatable manner. In the casing 92, an intake space 101 communicating with the intake port 95 and a liquid delivery space 102 communicating with the liquid delivery port 96 are formed. The proximal end opening 50A of the outer tubular shaft 50 is located in the intake space 101 in a rotatable manner.

The rotary operation portion 91 is a section operated by the operator with his/her finger to apply a torque to the outer tubular shaft 50. The rotary operation portion 91 is rotatably connected to the distal end part of the casing 92. The rotary operation portion 91 is fixed to the outer peripheral surface of the outer tubular shaft 50 at the proximal end part.

The drive unit 93 is, for example, a hollow motor. The drive unit 93 is rotated by a battery (not illustrated) or power supplied from the outside. The drive shaft 20 is fixed to a hollow drive rotor of the hollow motor. The drive unit 93 is not particularly limited in rotation speed and may rotate at, for example, 10,000 to 150,000 rpm, preferably 20,000 to 120,000 rpm. Note that the configuration of the drive unit 93 is not particularly limited. Since the drive unit 93 is a hollow motor, the guide wire lumen tube 40 and the guide wire can pass through the drive unit 93.

The intake port 95 conveys an object, liquid, or the like inside the intake space 101 to the outside. An intake drive source such as a pump or a syringe can also be connected to the intake port 95. From the liquid delivery port 96, a fluid can be delivered to the first lumen 57 of the outer tubular shaft 50 via the liquid delivery space 102.

The operation instruction portion 97 is a section operated by the operator to activate or stop the drive unit 93. The operation instruction portion 97 is located on the outer surface of the casing 92. The operation instruction portion 97 includes, for example, a push-button switch. The operation instruction portion 97 includes, for example, an operation start button 97A for instructing the start of operation and an operation stop button 97B for instructing the stop of the operation. The button for instructing the start of the operation and the button for instructing the stop may be common.

The control unit 120 includes a memory circuit and an arithmetic circuit. The memory circuit stores programs and various parameters. The arithmetic circuit is, for example, a central processing unit (CPU) and can read programs or various parameters from the memory circuit and perform arithmetic processing. The control unit 120 is, for example, a computer, a microcontroller, a microprocessor, or the like.

As illustrated in FIG. 4, the control unit 120 is instructed to start and stop the operation by the operation instruction portion 97, and controls the operation of the drive unit 93. In addition, the control unit 120 acquires information regarding the number of rotations of the drive unit 93 from the rotation detection unit 130.

The rotation detection unit 130 detects the number of rotations of the drive unit 93, the drive shaft 20, or the cutter 80. The rotation detection unit 130 may be included in the configuration of the control unit 120. The control unit 120 can monitor a current value applied to the hollow motor of the drive unit 93 and specify the number of rotations from the current value. The rotation detection unit 130 is not particularly limited and may be, for example, an optical tachometer or a magnetic tachometer.

Alternatively, in a case where the motor of the drive unit 93 is a brushless motor having a function of detecting the number of rotations (function as a Hall magnetic sensor), the motor of the drive unit 93 functions as the rotation detection unit 130.

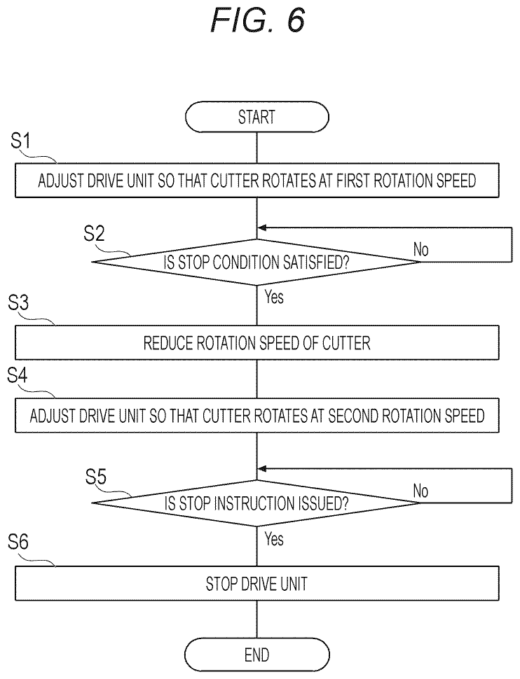

Next, the operation of the medical device 10 will be described with reference to a flowchart illustrated in FIG. 6. Here, a case where a calcified lesion S in the blood vessel is broken up and conveyed as illustrated in FIG. 5 will be described as an example.

First, the operator inserts the guide wire W into the blood vessel and brings the guide wire W to the vicinity of the lesion S. Next, the operator inserts the proximal end of the guide wire W into the guide wire lumen 41 of the medical device 10. Thereafter, the operator moves the cutter 80 of the medical device 10 to the vicinity of the lesion S using the guide wire W as a guide.

Next, the operator starts to deliver a liquid through the liquid delivery port 96, operates the operation instruction portion 97 after or while operating the handle 90 to orient the cutter 80 toward the lesion S, and starts the activation of the drive unit 93 (step S1). The wording “the cutter 80 is oriented toward the lesion S” means a state in which the lesion S is located on the distal side with respect to the cutter 80, and the cutter 80 comes into contact with the lesion S by moving straight toward the distal side along the central axis of rotation of the cutter 80.

When the operation instruction portion 97 is operated, the control unit 120 controls the drive unit 93 to rotate the cutter 80 at a preset first rotation speed (step S1). A prior operation at the first rotation speed is intended to eliminate the twist of the drive shaft 20 and the outer tubular shaft 50, and the cutter 80 does not cut the lesion S during this prior operation. The first rotation speed is low such as 400 rpm to 10,000 rpm, and is 5,000 rpm as an example. The first rotation speed may be higher. The first rotation speed may be high such as 10,000 rpm to 60,000 rpm. That is, the first rotation speed may be substantially the same as a second rotation speed described later.

Meanwhile, the drive shaft 20 and the outer tubular shaft 50 of the medical device 10 inserted into the meandering blood vessel may be twisted and may have accumulated strain. The twist of the drive shaft 20 increases when the friction between the drive shaft 20 and the outer tubular shaft 50 is high, and the drive shaft 20 is twisted greatly like a spring inside the outer tubular shaft 50. Therefore, when the twist of the drive shaft 20 is eliminated during the procedure, the orientation of the cutter 80 may be changed to an unintended direction.

In addition, the twist of the outer tubular shaft 50 increases when the friction between the outer peripheral surface of the outer tubular shaft 50 and the inner wall of the blood vessel is high, and the outer tubular shaft 50 is twisted greatly like a spring inside the blood vessel. Therefore, when the twist of the outer tubular shaft 50 is eliminated during the procedure, the orientation of the cutter 80 may be changed to an unintended direction.

On the other hand, when the drive shaft 20 and the cutter 80 rotate at the first rotation speed in the prior operation in which cutting is not performed, the contact between the inner peripheral surface of the outer tubular shaft 50 and the outer peripheral surface of the drive shaft 20 due to static frictional force is eliminated, and the twist accumulated in the drive shaft 20 and the outer tubular shaft 50 is eliminated. Further, when the outer tubular shaft 50 vibrates by the prior operation, the contact between the outer peripheral surface of the outer tubular shaft 50 and the inner wall of the blood vessel due to static frictional force is eliminated, and the twist accumulated in the outer tubular shaft 50 is eliminated. For this reason, at the time of a subsequent main operation at the second rotation speed in which cutting is performed, the twist has already been eliminated, and thus, it is possible to cut the desired position of the lesion S while appropriately adjusting the orientation of the cutter 80 in the intended direction using the rotary operation portion 91.

The control unit 120 continues the operation at the first rotation speed until a preset stop condition is satisfied. The control unit 120 determines whether or not the stop condition is satisfied (step S2), and when determining that the stop condition is satisfied, temporarily decreases the rotation speed (step S3). In step S3 for temporarily decreasing the rotation speed, the rotation may be completely stopped or decreased to a rotation speed lower than the first rotation speed without stopping the rotation. Alternatively, step S3 for temporarily decreasing the rotation speed may not be provided. That is, immediately after the prior operation at the first rotation speed, the main operation with high-speed rotation may be started without decreasing the rotation speed.

The stop condition of the prior operation is an elapsed time or the number of rotations. In a case where the stop condition of the prior operation is an elapsed time, the control unit 120 determines that the stop condition of the prior operation is satisfied when the elapsed time from the start of the operation at the first rotation speed reaches a preset threshold. The threshold of the elapsed time is preferably 0.01 seconds to 10 seconds.

When the stop condition of the prior operation is the number of rotations, the control unit 120 calculates the number of rotations from the start of the operation at the first rotation speed on the basis of a signal acquired from the rotation detection unit 130. The control unit 120 determines that the stop condition of the prior operation is satisfied when the calculated number of rotations reaches a preset threshold. The threshold of the number of rotations is preferably 5 rotations to 100 rotations.

After step S3 for temporarily decreasing the rotation speed (alternatively, after step S2 for determining whether or not the stop condition of the prior operation is satisfied), the control unit 120 controls the drive unit 93 to rotate the cutter 80 at the preset second rotation speed (step S4). The second rotation speed is a high rotation speed suitable for cutting by the cutter 80, and is preferably 10,000 rpm to 150,000 rpm. When the first rotation speed is a low speed not suitable for cutting, the second rotation speed is higher than the first rotation speed. When the first rotation speed is a high rotation speed suitable for cutting, the second rotation speed can be substantially the same as the first rotation speed.

When the drive unit 93 rotates, the cutter 80 rotates together with the drive shaft 20, and the cutter 80 can cut the lesion S. When the drive shaft 20 rotates, the conveyance coil 30 fixed on the drive shaft 20 generates a force for conveying the liquid or the object in the second lumen 59 to the proximal side as illustrated in FIG. 3. Thus, the cut fragment enters through the distal end opening 61 of the second lumen 59 of the outer tubular shaft 50 and is discharged as illustrated in FIG. 2.

The operator can change the position of the cutter 80 in the circumferential direction by operating the rotary operation portion 91 illustrated in FIG. 1. When the operator turns the rotary operation portion 91, the outer tubular shaft 50 fixed to the rotary operation portion 91 rotates. When the outer tubular shaft 50 rotates, the position and direction of the section of the outer tubular shaft 50 distal to the bending portion 56 change, and the position and orientation of the cutter 80 can be changed, as illustrated in FIG. 5. Therefore, the operator can perform the cutting operation while adjusting the position and orientation of the cutter 80 only by operating the rotary operation portion 91 without entirely rotating the handle 90 that is difficult to rotate greatly. Further, the operator moves the outer tubular shaft 50 back and forth along the longitudinal direction of the blood vessel by moving the entire handle 90 or the outer tubular shaft 50 exposed to the outside of the body. As a result, the lesion S can be cut along the longitudinal direction of the blood vessel by the cutter 80.

When the fluid is fed from the liquid delivery port 96 to the first lumen 57 of the outer tube shaft 50 via the liquid delivery space 102, the fluid is discharged to the first lumen 57 as illustrated in FIG. 3. The physiological saline delivered to the first lumen 57 moves in the distal direction and is released into the blood vessel through the side hole 58 formed at the distal end part of the outer layer 51.

A portion of the physiological saline released into the blood vessel enters the second lumen 59 together with blood and the cut object through the distal end opening 61 of the outer tubular shaft 50 as illustrated in FIGS. 2 and 5. The object and the liquid that have entered the second lumen 59 move in the second lumen 59 in the proximal direction. The object and the liquid that have entered the second lumen 59 are conveyed in the proximal direction by the conveyance coil 30 and discharged to an external waste liquid bag 100 through the intake port 95 of the handle 90 as illustrated in FIG. 1.

After the cutting and conveyance of the lesion S are completed, the operator operates the operation stop button 97B of the operation instruction portion 97. When receiving a signal instructing to stop the operation from the operation instruction portion 97 (step S5), the control unit 120 stops the drive unit 93 (step S6). Thus, the rotation of the drive shaft 20 is stopped. Thereafter, the operator removes the medical device 10 from the blood vessel, and the treatment is completed.

As described above, the medical device 10 according to the present embodiment is a medical device 10 for cutting an object in a blood vessel, the medical device 10 including: a drive shaft 20 that is rotatable; a cutter 80 disposed on the distal side with respect to the drive shaft 20; a drive unit 93 that rotates the drive shaft 20; an outer tubular shaft 50 that rotatably accommodates the drive shaft 20; and a control unit 120 that controls driving of the drive unit 93, in which the control unit 120 controls the drive unit 93 to rotate the cutter 80 at a preset first rotation speed (step S1), determines whether or not a stop condition of the prior operation at the first rotation speed is satisfied by the control unit 120 (step S2), and after determining that the stop condition of the prior operation is satisfied, controls the drive unit 93 to rotate the cutter 80 at a preset second rotation speed of 10,000 rpm or more and 150,000 rpm or less (step S4). Thus, the medical device 10 can eliminate the twist of the drive shaft 20 and the outer tubular shaft 50 by the prior operation, whereby the cutter 80 can be positioned in an appropriate orientation before performing a cutting operation.

The first rotation speed may be 400 rpm or more and 10,000 rpm or less. With this configuration, the prior operation is performed at a low speed, which makes the cutter 80 less likely to perform the cutting operation during the prior operation. Thus, safety can be improved.

The first rotation speed may be 10,000 rpm or more and 60,000 rpm or less. With this configuration, the first rotation speed is substantially the same as the second rotation speed, whereby the adjustment of the rotation speed is facilitated.

The stop condition of the prior operation may be an elapsed time of 0.01 seconds or more and 10 seconds or less. With this configuration, the stop condition of the prior operation can be easily set so that the twist of the drive shaft 20 and the outer tubular shaft 50 can be eliminated.

The stop condition of the prior operation may be the number of rotations of the cutter 80 that is five or more. With this configuration, the stop condition of the prior operation can be easily set so that the twist of the drive shaft 20 and the outer tubular shaft 50 can be eliminated.

The stop condition of the main rotation at the second rotation speed may be an elapsed time longer than the time of the prior operation. The stop condition of the main rotation at the second rotation speed is, for example, an elapsed time of 10 seconds to 60 seconds, preferably 30 seconds. With this configuration, it is possible to perform the main operation for cutting for sufficient time, whereby appropriate cutting can be performed. When the main rotation at the second rotation speed satisfies the elapsed time of the stop condition, the control unit 120 controls and stops the drive unit 93. Next, when a predetermined break time elapses, the control unit 120 can start the main rotation at the second rotation speed again. Then, the control unit 120 can repeat steps including performing the main rotation at the second rotation speed, stopping in a case where the stop condition is satisfied, and restarting the main operation in a case where the break time elapses. The operator manually operates the handle 90 and presses the operation stop button 97B of the operation instruction portion 97 to thereby stop the repetitive operation.

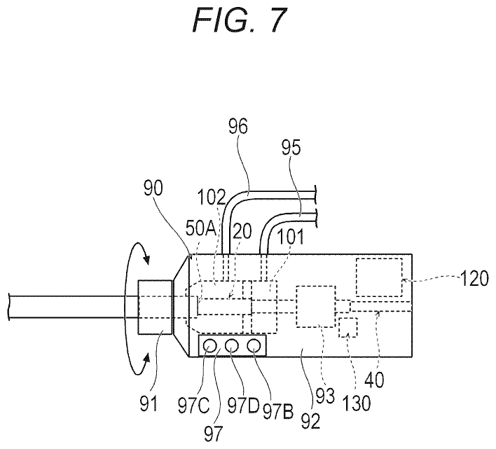

In the medical device 10 described above, whether or not the stop condition of the prior operation at the first rotation speed is satisfied or whether to start the main operation at the second rotation speed is determined by the control unit 120, but may be determined by the operator. To this end, as in a modification illustrated in FIG. 7, the operation instruction portion 97 may include a first operation start button 97C for instructing the start of rotation of the cutter 80 at the first rotation speed, a second operation start button 97D for instructing the start of rotation of the cutter 80 at the second rotation speed, and an operation stop button 97B for instructing the stop of the operation. The operator can press the first operation start button 97C when starting the prior operation at the first rotation speed (step S1). In addition, when determining that the stop condition of the prior operation is satisfied (step S2), the operator can press the operation stop button 97B to decrease or stop the rotation at the first rotation speed in the prior operation (step S3). The stop condition of the prior operation may be determined from the elapsed time or the number of rotations, but different conditions may be applied. For example, the operator may stop the prior operation when detecting that the twist of the drive shaft 20 or the outer tubular shaft 50 is eliminated from a cine image. Further, the operator can press the second operation start button 97D when starting the main operation at the second rotation speed. Note that the operator may press the second operation start button 97D without pressing the operation stop button 97B while the cutter 80 is rotating at the first rotation speed. With this process, the control unit 120 increases the rotation speed of the cutter 80 to the second rotation speed after reducing, stopping, or maintaining the rotation of the cutter 80 (step S4). The operator can press the operation stop button 97B to stop the main operation at the second rotation speed. Thus, the control unit 120 receives a stop instruction due to pressing of the operation stop button 97B (step S5), and stops the drive unit 93 (step S6).

As described above, the method for positioning the medical device 10 according to the present embodiment is a method for positioning the medical device 10 for cutting an object in a blood vessel, the medical device 10 including: a drive shaft 20 that is rotatable; a cutter 80 disposed on the distal side with respect to the drive shaft 20; a drive unit 93 that rotates the drive shaft 20; and an outer tubular shaft 50 that rotatably accommodates the drive shaft 20, the method including step S1 for bringing the cutter 80 close to the lesion S within the blood vessel, and then, rotating the cutter 80 at a preset first rotation speed, step S2 for determining whether or not a stop condition of the prior operation at the first rotation speed is satisfied, and step S4 for rotating the cutter 80 at a preset second rotation speed of 10,000 rpm or more and 150,000 rpm or less after it is determined that the stop condition of the prior operation is satisfied. Thus, with the method for positioning the medical device 10, the twist of the drive shaft 20 and the outer tubular shaft 50 can be eliminated by the prior operation, whereby the cutter 80 can be positioned in an appropriate orientation before performing a cutting operation.

The positioning method may include step S3 for reducing or stopping the rotation of the cutter 80 after it is determined that the stop condition of the prior operation is satisfied and before the cutter 80 is rotated at the second rotation speed. With this configuration, it is possible to confirm whether or not the twist of the drive shaft 20 and the outer tubular shaft 50 has been eliminated by the prior operation before the main operation for cutting is performed.

In the positioning method, the first rotation speed may be 400 rpm or more and 10,000 rpm or less. This configuration makes the cutter 80 less likely to perform the cutting operation during the prior operation, whereby safety can be improved.

In the positioning method, the first rotation speed may be 10,000 rpm or more and 60,000 rpm or less. With this configuration, the first rotation speed is substantially the same as the second rotation speed, whereby the adjustment of the rotation speed is facilitated.

In the positioning method, the stop condition of the prior operation may be an elapsed time of 0.01 seconds or more. With this configuration, the stop condition of the prior operation can be easily set so that the twist of the drive shaft 20 and the outer tubular shaft 50 can be eliminated.

In the positioning method, the stop condition of the prior operation may be the number of rotations of the cutter 80 that is five or more. With this configuration, the stop condition of the prior operation can be easily set so that the twist of the drive shaft 20 and the outer tubular shaft 50 can be eliminated.

In the positioning method, in step S1 for rotating the cutter 80 at the first rotation speed, the cutter 80 may be rotated at the first rotation speed after being oriented toward the lesion S in the blood vessel. The twist of the drive shaft 20 and the outer tube shaft 50 is eliminated after the cutter 80 is oriented to the lesion S in the blood vessel, whereby it is easy to recognize the elimination of the twist.

In the positioning method, in step S1 for rotating the cutter 80 at the first rotation speed, the cutter 80 may be rotated at the first rotation speed while being oriented toward the lesion S in the blood vessel. The twist of the drive shaft 20 and the outer tubular shaft 50 is eliminated while the cutter 80 is oriented to the lesion S in the blood vessel, whereby it is easy to appropriately adjust the position and the orientation of the cutter 80 that have changed after the elimination of the twist.

The medical device is not limited to the above embodiment, and various modifications may be made by those skilled in the art within the technical idea of the present invention. For example, the body lumen into which the medical device 10 is inserted is not limited to a blood vessel, and may be, for example, a vascular channel, a ureter, a bile duct, a fallopian tube, a hepatic duct, or the like.

The detailed description above describes embodiments of a medical device and operational method representing examples of the new medical device and operational method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A method involving positioning a medical device in a blood vessel and cutting an object in the blood vessel, the medical device including a drive shaft that is rotatable, a cutter disposed on a distal side of the drive shaft, a drive unit that rotates the drive shaft, and an outer tubular shaft that rotatably accommodates the drive shaft, the method comprising:
bringing the cutter close to a lesion within the blood vessel, and then, rotating the cutter at a first rotation speed that is set in advance;
determining whether or not a stop condition of a prior operation at the first rotation speed is satisfied; and
rotating the cutter at a second rotation speed that is set in advance after it is determined that the stop condition of the prior operation is satisfied, the second rotation speed being 10,000 rpm or more and 150,000 rpm or less.

2. The method according to claim 1, further comprising reducing or stopping the rotation of the cutter after it is determined that the stop condition is satisfied and before the cutter is rotated at the second rotation speed.

3. The method according to claim 1, wherein the first rotation speed is 400 rpm or more and 10,000 rpm or less.

4. The method according to claim 1, wherein the first rotation speed is 10,000 rpm or more and 60,000 rpm or less.

5. The method according to claim 1, wherein the stop condition is an elapsed time of 0.01 seconds or more.

6. The method according to claim 1, wherein the stop condition is a number of rotations of the cutter that is five or more.

7. The method according to claim 1, wherein, in the rotating of the cutter at the first rotation speed, the cutter is rotated at the first rotation speed after the cutter is oriented to the lesion within the blood vessel.

8. The method according to claim 1, wherein, in the rotating of the cutter at the first rotation speed, the cutter is rotated at the first rotation speed as the cutter is oriented to the lesion within the blood vessel.

9. A method comprising:
positioning a cutter in a blood vessel, the cutter being connected to a rotatable drive shaft so that rotation of the drive shaft results in rotation of the cutter;
moving the cutter towards a lesion in the blood vessel;
starting rotation of the cutter and continuing rotation of the cutter during a first operation at a first rotation speed through rotation of the drive shaft while the lesion is located distal of the cutter so that the cutter does not cut the lesion;
determining that a stop condition of the first operation at the first rotation speed is satisfied; and
rotating the cutter at a preset second rotation speed through rotation of the drive shaft while the cutter is in contact with the lesion so that the lesion is cut by rotation of the cutter at the second rotation speed, the rotating of the cutter at the preset second rotation speed occurring after determining that the stop condition of the first operation at the first rotation speed has been satisfied.

10. The method according to claim 9, wherein the rotating of the cutter at the preset second rotation speed while the cutter is in contact with the lesion includes rotating the cutter at the preset second rotation speed that is 10,000 rpm or more and 150,000 rpm or less.

11. The method according to claim 9, wherein the determining that the stop condition of the first operation at the first rotation speed is satisfied includes determining that a time of 0.01 seconds or more and 10 seconds or less has elapsed since the starting of the rotation of the cutter.

12. The method according to claim 9, wherein the determining that the stop condition of the first operation at the first rotation speed is satisfied includes determining that the cutter has rotated at least five times since the starting of the rotation of the cutter.

13. The method according to claim 9, wherein the starting of the rotation of the cutter includes operating an operation start button, the rotating of the cutter at the preset second rotation speed occurring automatically after that the stop condition has been satisfied.

14. The method according to claim 9, wherein the rotating of the cutter at the preset second rotation speed after determining that the stop condition of the first operation at the first rotation speed has been satisfied includes rotating the cutter at the preset second rotation speed that is greater than the first rotation speed.

15. A medical device for cutting an object in a blood vessel, the medical device comprising:
a rotatable drive shaft;
a cutter disposed on a distal side with respect to the drive shaft;
a drive unit that rotates the drive shaft;
an outer tube shaft that rotatably accommodates the drive shaft;
a control unit that controls driving of the drive unit; and
the control unit being configured to: i) control the drive unit to rotate the cutter at a first rotation speed that is set in advance; ii) determines whether or not a stop condition at the first rotation speed is satisfied; and iii) rotates the cutter at a second rotation speed that is set in advance after determining that the stop condition is satisfied, the second rotation speed being 10,000 rpm or more and 150,000 rpm or less.

16. The medical device according to claim 15, wherein the first rotation speed is 400 rpm or more and 10,000 rpm or less.

17. The medical device according to claim 15, wherein the first rotation speed is 10,000 rpm or more and 60,000 rpm or less.

18. The medical device according to claim 15, wherein the stop condition is an elapsed time of 0.01 seconds or more and 10 seconds or less.

19. The medical device according to claim 15, wherein the stop condition is a number of rotations of the cutter that is five or more.

20. The medical device according to claim 15, wherein a stop condition of main rotation at the second rotation speed is an elapsed time longer than a time of rotating the cutter at the first rotation speed.
